(19) United States
(12) Patent Application Publication
(10) Pub. No.: US 2013/0303881 A1
(43) Pub. Date: Nov. 14, 2013

(54) EQUIPMENT OBJECT FOR A COMBINATION IMAGING SYSTEM

(71) Applicant: SIEMENS AKTIENGESELLSHAFT, Munich (DE)

(72) Inventors: Martin HEMMERLEIN, Bamberg (DE); Ralf LADEBECK, Erlangen (DE)

(21) Appl. No.: 13/873,357

(22) Filed: Apr. 30, 2013

(30) Foreign Application Priority Data
May 9, 2012 (DE) 102012207677.8

(51) Int. Cl. A61B 5/00

(52) U.S. Cl. CPC A61B 5/0035
USPC 600/411

(57) ABSTRACT

An equipment object is provided for a combination imaging system and can be positioned in a measurement chamber. The equipment object includes a radionuclide imaging device and a magnetic resonance imaging device. In its peripheral region the equipment object further includes an image-critical function component which has an average radionuclide emission radiation attenuation value that reaches at least a specified attenuation limit value of 30% in relation to a first defined minimum cross-sectional area of 30 mm², and/or wherein the equipment object is so configured that an average radionuclide emission radiation attenuation value relating to a second defined minimum cross-sectional area of 400 mm² of the equipment object reaches at most a central attenuation limit value of 15% in an overall spatially central region of the equipment object.

### PRIORITY STATEMENT

The present application hereby claims priority under 35 U.S.C. §119 to German patent application number DE 102012207677.8 filed May 9, 2012, the entire contents of which are hereby incorporated herein by reference.

### FIELD

At least one embodiment of the present invention generally relates to a method for designing an equipment object for a combination imaging system comprising a magnetic resonance imaging unit and a radionuclide imaging unit, a method for designing a combination imaging system, an equipment object for a combination imaging system, and/or a combination imaging system.

### BACKGROUND

Imaging methods for representing examination objects, in particular for determining inter alia the properties, arrangement and extent of materials, are widely used in medical applications in particular.

There now exists a wide range of imaging systems which can be used to generate recordings of the interior of the body of a patient. These include e.g. magnetic resonance tomography devices and computer tomographs, by means of which anatomical images can be generated. There also exist radionuclide emission tomography recording devices such as PET systems (PET=Positron Emission Tomography) and SPECT systems (SPECT=Single Photon Emission Computer Tomography), in which small quantities of substances to which radioactive matter has been added, so-called “tracers”, are injected into the human body in order to identify various metabolisms in the body by means of measuring the radioactive radiation. The quantity of the injected substance is extremely small and lies in the subphysiological range. Therefore the metabolic processes to be examined are not influenced and no toxic reactions occur. The radiation of the injected substance or the photon radiation that is generated by the injected substance is registered by means of radiation detectors and an image is generated therefrom. In this case, the radionuclide image generation is based on the analysis of count rates and trajectories of the photons or coincidentally measured photon pairs that are generated by the injected radionuclide. The determination of count rates and trajectories allows a reverse calculation of the condition of the examination object, and essentially defines the image information that is obtained by means of the radionuclide-based radiation. The tracer accumulates in specific organs and/or tumors, thereby allowing the metabolisms to be diagnosed very effectively and, in particular, allowing tumors and metastases in the surrounding tissue to be identified very easily and precisely. Such methods also allow the perfusion of the heart muscle to be evaluated, for example.

Whereas on one hand magnetic resonance tomography allows the generation of a relatively well spatially resolved image data record in which it is particularly easy to recognize anatomical structures such as specific organs, for example, PET and SPECT on the other hand are used to generate images in which it is particularly easy to identify specific pathological changes while anatomical structures are generally depicted poorly or not at all. As a result of this, provision is increasingly made for capturing both magnetic resonance images and radionuclide emission tomography image data relating to an examination object, these being adapted to each other such that they can be superimposed in a spatially accurate manner to form a single image. The geometric adaptation of the image data of the individual images, which is also known as “registering” the images and is required for said superimposition, involves considerable computing effort. Consequently, there now exist combined imaging systems, also referred to in the context of an embodiment of the invention as “combination imaging systems”, which comprise both a magnetic resonance recording device and a radionuclide emission tomography recording device. Here too, the magnetic resonance images and the radionuclide emission tomography image data are initially processed entirely separately and then superimposed. However, these systems have the advantage that the images, having been produced in the same system and in (almost) the same position of the examination object, are already registered by virtue of the hardware and are therefore easier to superimpose with spatial accuracy.

For this purpose, the examination object is arranged in a shared measurement chamber of the combination imaging system or combination tomograph, said measurement chamber being used for the different imaging methods simultaneously. With regard to the radionuclide-based image generation, which is performed by means of PET or SPECT tomographs as cited in the introduction, this however gives rise to the problem that components of the magnetic resonance tomograph, which are combined in the same device and preferably used simultaneously, may be arranged between an examination object and the radiation detector cited in the introduction, and these components then impede or change the radionuclide-based image acquisition.

The components which are arranged between the examination object and the radiation detector give rise to an attenuation of the radionuclide-based radiation. This results in changed count rates and changed trajectories of corresponding photons, such that interference or variation in the quality of the image information inevitably results in many cases.

### SUMMARY

An embodiment of the present invention is therefore to moderate this problem.

An embodiment of the invention is directed to an equipment object, a combination imaging system, a method for designing an equipment object, and/or a method for designing a combination imaging system.

An embodiment of the invention is directed to an equipment object for a combination imaging system comprising a radionuclide imaging device such as e.g. a PET (Positron Emission Tomography) or SPECT (Single Electron Emission Computer Tomography) imaging device and a magnetic resonance imaging device, wherein the equipment object is arranged or can be positioned as standard in a measurement chamber of the combination imaging system, between a radionuclide radiation source and a radiation detector unit for the radionuclide emission radiation in the combination imaging system.

An embodiment of an inventive method for designing an equipment object comprises the step of selecting a first image-critical function component from the group of all function components of the equipment object on the basis of its attenuation value for radionuclide emission radiation and/or its attenuation correction factor. A further step of an embodiment of the inventive method comprises the arrangement of the selected first image-critical function component or at least parts of said image-critical function component in a peripheral region of the equipment object.

### BRIEF DESCRIPTION OF THE DRAWINGS

The invention is explained again in greater detail below with reference to the appended figures and example embodiments. In this case, identical components are denoted by identical reference signs in the figures, in which:

FIG. 1 shows the schematic structure of a combination imaging system in a perspective view,

FIG. 2 shows the schematic structure of a combination imaging system in a cross-sectional representation,

FIG. 3 shows a diagram for determining an attenuation correction factor for a plurality of function components of a patient table according to the prior art as shown beneath the diagram,

FIG. 4 schematically shows a first example embodiment of the arrangement of function components on an equipment object,

FIG. 5 shows a cross-sectional illustration explaining the effect of an inventive rearrangement or transfer of a first function component on a patient table from a first position to a second position in accordance with a first example embodiment,

FIG. 6 shows a schematic illustration of the shade surfaces produced on a radiation detector unit by the function component on a patient table as per FIG. 5 in the first position and the second position,

FIG. 7 shows a cross-sectional illustration explaining the effect of an inventive rearrangement or transfer of a second function component on a patient table from a first position to a second position in accordance with a second example embodiment, and

FIG. 8 shows an illustration explaining the effect of an example of an inventive rearrangement or transfer of function components in a local coil, in a plan view of the local coil.

### DETAILED DESCRIPTION OF THE EXAMPLE EMBODIMENTS

The present invention will be further described in detail in conjunction with the accompanying drawings and embodiments. It should be understood that the particular embodiments described herein are only used to illustrate the present invention but not to limit the present invention.

Accordingly, while example embodiments of the invention are capable of various modifications and alternative forms, embodiments thereof are shown by way of example in the drawings and will herein be described in detail. It should be understood, however, that there is no intent to limit example embodiments of the present invention to the particular forms disclosed. On the contrary, example embodiments are to cover all modifications, equivalents, and alternatives falling within the scope of the invention. Like numbers refer to like elements throughout the description of the figures.

Specific structural and functional details disclosed herein are merely representative for purposes of describing example embodiments of the present invention. This invention may, however, be embodied in many alternate forms and should not be construed as limited to only the embodiments set forth herein.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items.

It will be understood that when an element is referred to as being “connected,” or “coupled,” to another element, it can be directly connected or coupled to the other element or intervening elements may be present. In contrast, when an element is referred to as being “directly connected,” or “directly coupled,” to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

Although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

An embodiment of the invention is directed to an equipment object for a combination imaging system comprising a radionuclide imaging device such as e.g. a PET (Positron Emission Tomography) or SPECT (Single Electron Emission Computer Tomography) imaging device and a magnetic resonance imaging device, wherein the equipment object is arranged or can be positioned as standard in a measurement chamber of the combination imaging system, between a radionuclide radiation source and a radiation detector unit for the radionuclide emission radiation in the combination imaging system.

In this case, an “equipment object” is understood to mean an object which is necessarily, ordinarily or optionally part of the equipment of the combination imaging system, and provides or enhances said system with a specific functionality. For example, the equipment object can be a mobile patient table which is used to support and move and position the examination object in the measurement chamber. A further example of an equipment object is e.g. a local coil which contributes to the generation and/or reception of magnetic resonance signals in the combination imaging system. In order to improve a signal to noise ratio, these local transmit and receive antennas are often arranged in the immediate vicinity of the examination object and therefore immediately adjacent to (i.e. in particular at minimal distance from) the radiation source. Since it is a particular advantage of the combination imaging system that images are acquired simultaneously or almost simultaneously by the different imaging methods, it is not possible to remove these equipment objects from the measurement chamber for the purpose of radionuclide-based image acquisition during standard operation.

The field of view of the radiation detector unit, i.e. the spatial region that can be captured by the radiation detector unit and hence the “vision” of the detector, is restricted by the equipment object. This also applies to a range of further inventive equipment objects which are described in greater detail below.

The equipment object according to an embodiment of the invention typically comprises a range of function components, each having a specific functionality and thus contributing to the overall functionality of the equipment object. In the case of the mobile patient table the function components can be rails for moving the table, for example, and in the case of the local coil such function components can be e.g. individual electronic components such as shielded printed circuit boards, for example.

An embodiment of the invention is based inter alia on the idea of improving the radionuclide-based imaging by way of an optimized arrangement, as described below in greater detail, of “image-critical function components” of the equipment object, such that a field of view of the radiation detector unit is increased in relation to said image-critical function components. In the context of embodiments of the invention, function components are designated and identified as “image-critical” if their interaction with the radionuclide emission radiation exceeds predetermined limit values. They can be identified in the context of an embodiment of the invention in a wide diversity of ways based on their transmission properties and/or scattering properties, based on an attenuation value, based on a shade angle or an associated shade surface which the image-critical component produces on the radiation detector unit for radionuclide emission radiation, and/or also based on compensation measures that are required in the combination imaging system, such as the arithmetic correction of recorded count rates by means of a so-called “attenuation correction factor” (ATF), for example. In particular, it is feasible to combine some or all of these parameters for the purpose of identifying image-critical function components.

In order to optimize the field of view of the radiation detector unit, the equipment object is inventively so configured as to comprise in its peripheral region an image-critical function component which has an average radionuclide emission radiation attenuation value that reaches at least a specified attenuation limit value of 30% and preferably 50% in relation to a first defined minimum cross-sectional area of 30 mm2 and particularly preferably 45 mm2 of said function component (the attenuation limit value relating likewise to the defined minimum cross-sectional area). As a result of the limit values specified thus, e.g. individual capacitors such as those required in a local coil are still not regarded as critical function components. In the context of embodiments of the invention, a function component is arranged in a peripheral region if a function component lies for the most part within the peripheral region.

In this case, a peripheral region of the equipment object is understood in the context of embodiments of the invention to be a spatial region which is contiguous with a periphery of the equipment object and has a predetermined peripheral region breadth. In this case, the peripheral region comprises that region of the equipment object which, in the case of a standard arrangement of the equipment object in the combination imaging system, comes closest to a surface of the radiation detector unit. In the context of embodiments of the invention, the peripheral region breadth is disposed along the periphery, starting in each case from the point or from a line or surface which comes closest to a surface of the radiation detector unit. In the case of a patient table, e.g. the side surfaces (or the edges) of the patient table are usually closest to the radiation detector unit. In the case of a patient table, the peripheral region therefore extends each case from a point on the side surface, or a line running through the point and along the side surface, towards the center of the patient table. Similarly, the peripheral region of a flat local coil, in particular a spine coil, extends from the narrow side or edge of the coil inwards towards the center of the local coil. In the context of embodiments of the invention, the lateral peripheral region is preferably understood (being thereby defined) to lie on or under the lateral peripheral regions of the patient body or even (partially) laterally beyond the patient body, assuming a standard positioning of the equipment object and a customary positioning of a patient in a prone or supine position in the measurement chamber. The lateral peripheral region of a patient table or a spine coil is therefore a laterally outermost strip of the table or coil. The peripheral region breadth amounts to at most a predetermined fraction, preferably a fifth, more preferably an eighth and most preferably a tenth of the volume of the equipment object.

In the case of an equipment object which exceeds the dimensions of the contour of an examination object, the peripheral region in which the image-critical function component may lie is preferably selected such that, in the case of a standard arrangement of the equipment object in the combination imaging system, the peripheral region is located outside of a projection of the examination object onto the equipment object in the direction of the closest surface of the equipment object. In the case of a patient table, this can be e.g. that region of the patient table which is located outside of a typical contour surface of a patient on the table surface. This peripheral region generally contains fewer so-called “relevant lines of response”. These include straight connection lines between image points of the annular PET detector, which lines run through the examination object and along which the two photons resulting from an annihilation event fly away from each other in opposite directions and can then be measured quasi coincidentally at the image points in order to identify and localize an event.

The above cited inventive average radionuclide emission radiation attenuation value relates to a minimum cross-sectional area. The average radionuclide emission radiation attenuation value corresponds in this case to a normalized percental change of a count rate of radionuclide emission quanta (photons), which is conditional upon the radiographic penetration of the equipment object in the region of this defined minimum cross-sectional area by radionuclide emission radiation of a homogeneous density from a defined spatial direction. The normalization of the change in the count rate is effected per time unit, while the percental change is determined relative to the initial radionuclide emission radiation arriving at the equipment object in the region of the minimum cross-sectional area from the individual spatial direction.

If the attenuation value for the image-critical function component is determined in relation to the first minimum cross-sectional area of the function component, this ensures that function components are only classified as image-critical if they have an effect on a predetermined number of image points of the radiation detector unit, i.e. if they contribute significantly to a particularly poor field of view of the radiation detector.

The inventive equipment object of at least one embodiment is alternatively or additionally configured such that an average radionuclide emission radiation attenuation value relating to a second defined minimum cross-sectional area of 400 mm2 of the equipment object reaches at most a central attenuation limit value of 15% in an overall spatially central region of the equipment object. In the context of embodiments of the invention, the term “spatially central region” relates to a spatial region in or on the equipment object, which spatial region is surrounded by and/or adjoins the “peripheral region” and defines the remaining volume of the equipment object relative to the peripheral region.

If the attenuation value for the spatially central region is determined in relation to said second minimum cross-sectional area of the equipment object, this ensures that the spatially central region is as free as possible of regions that significantly restrict the field of view of the radiation detector unit.

A combination imaging device according to an embodiment of the invention features a radionuclide imaging device (PET or SPECT) comprising a radiation detector unit for radionuclide emission radiation, a magnetic resonance imaging device, and an equipment object which is arranged in a measurement chamber of the combination imaging system between an examination object and the radiation detector unit.

This equipment object is constructed in the inventive manner described above.

As an alternative or in combination with the above, the image-critical function component can be identified by way of an “attenuation correction factor” in the case of a standard arrangement of the equipment object in the combination imaging system. This means that the equipment object arranged in the inventive combination imaging system comprises, in its peripheral region, an image-critical function component which produces an attenuation correction factor that reaches a correction limit value of at least 1.5 in the combination imaging system.

The attenuation correction factor is a scaling factor which is determined for each line of response of the radiation detector unit. By combining the scaling factor with a radionuclide radiation density value (count rate of the detectors for this line of response) which has been attenuated by the equipment object or the function component and has been determined for a line of response, it is possible to determine the initial radionuclide radiation density, i.e. the radionuclide radiation density without the equipment object in the beam path. It is correspondingly possible to specify a correction limit value for the attenuation correction factor, wherein a function component of the equipment object is considered to be image-critical if said correction limit value is exceeded. This definition of the limit value can be done on the basis of e.g. a point radiation source or a cylindrical radiation source (the cylinder axis being oriented in the longitudinal direction of the imaging system) that is arranged in the usual region in which the examination object is also located during a measurement. This “normal radiation source” for the limit value definition is preferably arranged at a distance of approximately 15 to 30 cm, preferably approximately 20 cm, from the surface of the equipment object or the image-critical function component. The attenuation correction factor which is necessitated by an equipment object or function component and/or the correction limit value is further determined relative to e.g. an annular radiation detector unit that surrounds the equipment object and has a diameter of 50-70 cm, preferably approximately 65 cm. The position of the point radiation source or cylindrical radiation source preferably corresponds to the center point or center line (longitudinal axis or axis or rotation) of the annular radiation detector unit, and the radiation source is particularly preferably a cylindrical phantom object which has a length of 30 cm and a diameter of 20 cm, and whose longitudinal axis corresponds to the center line or axis of rotation of the imaging system.

In a similar manner, this arrangement can also be used in the context of embodiments of the invention to determine an attenuation correction factor in relation to the second minimum cross-sectional area of the equipment object.

Furthermore, the equipment object is alternatively or additionally configured such that the attenuation correction factor reaches at most a defined central correction limit value in relation to the overall spatially central region of the equipment object. In the context of embodiments of the invention, the central correction limit value is preferably 1.2 and particularly preferably 1.3.

An embodiment of the invention makes use of a number of insights in this case, in order in particular to ensure that a “shade angle” or associated “shade surface” observed in the context of embodiments of the invention for radionuclide emission radiation is as small as possible relative to a corresponding radiation detector unit for radionuclide emission radiation, and thereby to allow the corresponding image information to be captured with minimum impairment and to improve the field of view of the radiation detector unit.

The “shade surface” observed in the context of embodiments of the invention can be determined e.g. relative to a point radionuclide emission radiation source which is located at a defined distance (preferably approximately 20 cm) from the surface of the equipment object, or is arranged in the center of the measurement chamber in the case of a standard arrangement in the combination imaging system.

The radiation angle (of the point radionuclide emission radiation source) that is masked by the function component is referred to below as the so-called “shade angle”. The “shade surface” is therefore derived from the projection of the shade angle onto the radiation detector unit.

In a combination imaging system, a radiation source for radionuclide emission radiation, i.e. a phantom or an examination object, is usually arranged centrally (in a topological center) in a measurement chamber which is essentially surrounded annularly by a radiation detector unit for radionuclide emission radiation. As a result of transferring image-critical function components relative to the center of the radiation detector unit or of the measurement chamber, which essentially corresponds to the center of the radiation source during operation, there is a change in the shade angle for radionuclide emission radiation relative to the radiation detector unit, said shade angle being produced by the image-critical function component. The further away this function component is arranged relative to the center, or the closer this function component is arranged relative to a radiation detector, the smaller its associated shade angle relative to the annular radiation detector unit.

If the image-critical function component is arranged in a peripheral region of the equipment object, the associated shade angle can be minimized.

An embodiment of an inventive method for designing an equipment object comprises the step of selecting a first image-critical function component from the group of all function components of the equipment object on the basis of its attenuation value for radionuclide emission radiation and/or its attenuation correction factor. A further step of an embodiment of the inventive method comprises the arrangement of the selected first image-critical function component or at least parts of said image-critical function component in a peripheral region of the equipment object.

An embodiment of an inventive method for designing a combination imaging system comprising a radionuclide imaging device, a magnetic resonance imaging device and an equipment object, which is arranged as standard between an examination object and a radiation detector unit for radionuclide emission radiation, correspondingly comprises the steps: selecting an image-critical function component of the equipment object on the basis of an attenuation value and/or an attenuation correction factor and arranging the function component or at least parts of the image-critical function component in a peripheral region of the equipment object.

An embodiment of the inventive design method for the equipment object or the combination imaging system comprises both the planning and the production of the equipment object or the combination imaging system in this case.

Further particularly advantageous embodiments and developments of the invention are derived from the dependent claims and from the following description, wherein the independent claims in one class of claim can also be developed in a similar way to the dependent claims in another class of claim.

In a development of an embodiment of the invention, the image-critical function component itself can be optimized in respect of its associated shade angle. This can be achieved e.g. by “partitioning” the image-critical function component into an image-critical function assembly comprising a plurality of smaller function components, which when combined have the functionality of the image-critical function component. The function assembly or the image-critical function component can therefore be arranged such that a remaining central region of the equipment object, relative to the peripheral region, is free of image-critical function components or image-critical function assemblies.

By virtue of this arrangement of the function assembly, which is now distributed over a larger surface area, it is possible to ensure that the image-critical shade angle for function components of the function assembly is minimal or optimal if the attenuation value of the function assembly is less than or at most equal to that of an “unpartitioned” critical function component.

In a particular example embodiment, the method for designing the equipment object or the combination imaging device therefore comprises the provision of a further, second function component. In this case, partitioning can be effected in respect of an overall capacity or overall functionality of the image-critical first function component, such that only in combination do the first and second function components achieve an overall capacity or overall functionality of the “unpartitioned” image-critical function component, which is required or specified for the operation of the equipment component in the combination imaging system. The resulting first and second function components consequently form a function assembly as described.

In a development of this idea, the first and second function components can also be so configured as to be functionally identical, wherein an overall functionality of a function assembly is preferably achieved again by virtue of the preferably parallel interaction of the first and second function components, i.e. the first and second function components are so configured as to execute identical partial functionalities of an overall functionality in parallel.

In a further method step, the second function component that has been provided is preferably so arranged as to be spatially separate from the first function component, in particular such that they are essentially on opposite sides of the equipment object and particularly preferably in a peripheral region thereof.

It is thereby possible to achieve a minimal shade angle, relative to the radiation detector unit, of the first and second function components that have been provided.

This advantage can also be achieved in particular by way of an equipment object that comprises a plurality of function components in a peripheral region of the equipment object and preferably essentially on opposite sides thereof, wherein said function components are essentially identical in function and/or have an overall functionality that results from their functional combination.

In an equipment object, provision is preferably made for arranging in the peripheral region precisely that image-critical function component which on one hand can be transferred, i.e. does not necessarily have to be arranged in the undesirable region and/or has sufficient space in the peripheral region, and on the other hand has the highest attenuation value or produces the highest attenuation correction factor of all function components of the equipment object. If possible, further function components can then be transferred into the peripheral region according to this rule.

The equipment object may comprise a plurality of image-critical function components, for example, some of which however require a fixed arrangement in the equipment object due to their function. In order nonetheless to achieve an optimization of the radionuclide-based image information, it is possible to specify or select the image-critical function component that is at least partially transferable in terms of design, such that an optimal arrangement of the function component in the peripheral region of the equipment object can be selected accordingly. The term “partially transferable” in this case also includes the partitioning of the image-critical function component when forming a function assembly.

The material that is chosen for parts of the equipment object also has an influence on transmission properties and/or scattering properties in respect of radionuclide emission radiation. Metallic components for example, but also components made of certain plastics such as glass fiber reinforced resins, have a high attenuation value and may necessitate a high attenuation correction factor.

In a particular example embodiment, function components having metallic portions are identified as image-critical function components and arranged according to an embodiment of the invention. Due to their high attenuation value, it is therefore particularly advantageous to the image information capture if these components are arranged in a peripheral region of the equipment object as per an embodiment of the invention. Provision is preferably made for identifying as image-critical and arranging as per an embodiment of the invention those function components whose metallic portions have a proportional cross-sectional area of at least 20%, particularly preferably at least 30%, and most preferably at least 40% relative to the first minimum cross-sectional area.

In an example development of an embodiment of the invention, the equipment object is so configured as to be essentially flat and therefore has a face side and a narrow side, wherein the equipment object can be inscribed in a cuboid having a face side and a narrow side such that the equipment object directly adjoins each side of the cuboid. In this case, the edges of the cuboid are considered to belong to each of the adjoining sides. It is possible in this case to achieve a particularly advantageous minimization of the shade angle by arranging the image-critical function component in the region of the narrow side of the equipment object. For example, the image-critical function component can be arranged immediately at the narrow side, e.g. fastened to the narrow side. Alternatively, the function component can also be arranged at a short distance from the narrow side. A distance is “short” in this context if it is less than the width of the image-critical function component in a breadth direction parallel to the imaginary shortest line between the function component and the respective narrow side. The distance from the radiation detector, and the associated shade angle of function components as described above, is thus minimized.

In an embodiment of the invention, the equipment object comprises a device item which is integrated in the measurement chamber of the imaging system, in particular a support system for an examination object, preferably a patient table of the combination imaging system.

Furthermore, the equipment object can also comprise a function ancillary unit which is optionally placed in the measurement chamber depending on the examination. In a particularly preferred embodiment, the function ancillary unit can be a local coil for receiving magnetic resonance signals and/or for transmitting high-frequency signals.

The image-critical function component can preferably be selected from a group of mechanical function components and/or electrical function components of the device item or the function ancillary unit respectively.

The mechanical components can comprise e.g. mechanical drive components, guide components such as e.g. a gear rack, metal bearings, particularly ball bearings, and mechanical strengthening components such as e.g. glass fiber reinforced components. The electrical components can comprise e.g. shielding devices, in particular sheath wave traps or bazookas, circuit boards, cable sections, electrical modules, in particular discrete and/or integrated modules such as amplifier circuits, for example.

The cited function components contribute significantly in each case to the attenuation of the radionuclide emission radiation in a device item or a function ancillary unit, and therefore a selection and transfer of these image-critical function components into the peripheral region of a function ancillary unit or a device item can, by virtue of minimizing the shade angle of the function component, optimize the attenuation of the radionuclide emission radiation relative to the radiation detector unit due to the function ancillary unit or the device item. An optimization is therefore achieved in relation to the field of view of the radiation detector unit for radionuclide emission radiation in the combination imaging system.

For a predetermined size of an essentially flat equipment object, an optimization in relation to the field of view of the equipment object is established in particular if the ratio between a shade surface of the function component and a shade surface produced by the face side of the equipment object is smaller than a predetermined surface ratio, which can be specified as 1:10, more preferably as 1:9 and most preferably as 1:8, for example. This means that the projection of the image-critical function component from the radiation source onto the detector surface does not exceed the predetermined surface ratio relative to the projection of the face side onto the detector surface.

In a development of the combination imaging system, the image-critical function component on the radiation detector unit is assigned a shade surface which corresponds to or exceeds a predetermined number of contiguous image points on the radiation detector unit for radionuclide emission radiation. In the context of embodiments of the invention, the predetermined number of image points is preferably specified as 3×3 contiguous image points, i.e. an image dot matrix of corresponding size.

In a design method of the equipment component, it is possible to achieve a reduction in the shade surface by transferring the selected or provided image-critical function component in the direction of the radiation detector unit, preferably until the number of contiguous image points shaded by the function component falls below a “permissible number of image points” of preferably 5×5 image points if possible.

In a development of this idea for achieving a minimal shade angle, a method for designing an equipment object can also provide for e.g. extending the dimension of the equipment object (relative to a conventional design form used before the inventive optimization of the function components) in a spatial direction, such that the equipment object is then so configured as to be flatter than would be the case without the optimization. The function component that is provided or selected can be arranged in the region of the extension, e.g. at a distance from further function components, preferably in a peripheral region, particularly preferably in the region of the narrow side of the equipment object. In a particularly preferred embodiment, separate fastening elements are arranged on the equipment object, e.g. supports or holders, in order to fasten the function components to the equipment object at a distance. The distance of the function component from the equipment object, or the dimension of the support or holder in the distance direction, is preferably at least twice (particularly preferably at least three times) the dimensions of the function components in the direction of the distance.

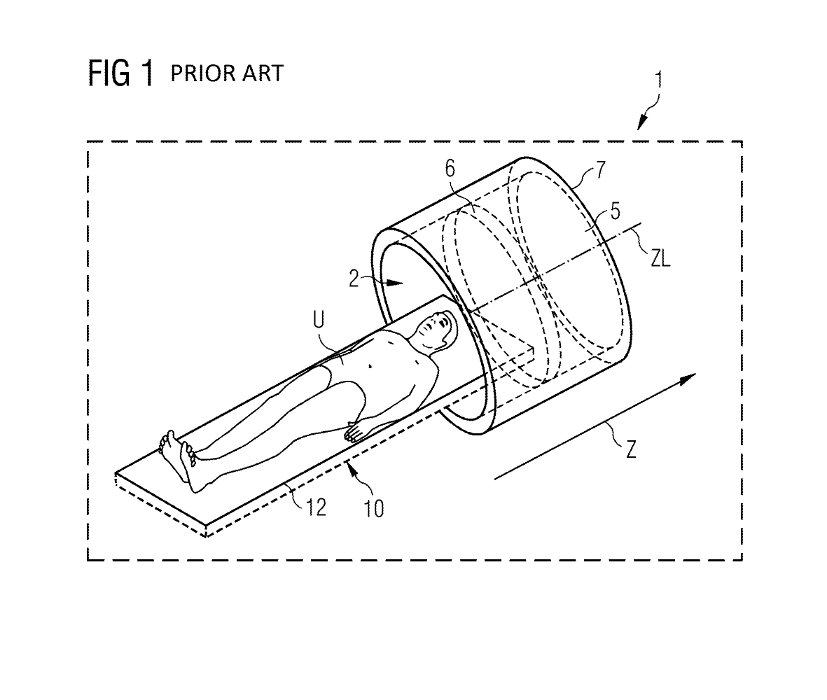

FIG. 1 schematically shows the structure of a combination imaging system 1 comprising a radionuclide imaging device 5 and a magnetic resonance imaging device 7. The radionuclide imaging device 5 is embodied as a PET imaging device 5 in this case, though it is equally conceivable for the radionuclide imaging device 5 to be developed as a SPECT imaging device. In addition to further components known to a person skilled in the art, the PET imaging device features a radiation detector unit 6 for positron recombination radiation having an energy of approximately 511 keV. In this case, the preferred embodiment comprises scintillation crystals, which convert the high-energy PET radiation into photons that can be captured by photodiodes. Annihilation of one positron and one electron (pairing) results in the generation of two photons, each having an energy of approximately 511 keV, whose trajectories run in opposite directions. These photon pairs can be measured coincidentally by way of the PET radiation detector 6, thereby allowing an inverse calculation of the trajectories and hence a spatial determination of the point of origin of the detected photon pairs in an examination object U. This inverse calculation allows the spatial concentration of the tracer in the examination object U to be determined. In conjunction with the image information from the magnetic resonance imaging device 7, it is therefore possible to acquire high-resolution detailed combination images of the examination object U, in which the tracer concentration can be identified in its anatomical surroundings.

In the example embodiment, the radiation detector unit 6 is arranged annularly around a central axis ZL of a measurement chamber 2 of the combination imaging system 1, the central axis ZL being oriented essentially parallel with a spatial direction z that corresponds to the alignment of a basic magnetic field of the combination imaging system 1, wherein basic magnetic field is explained further below. The annular arrangement allows an essentially identical distance between an examination object U, which is arranged in the center or in the region of the central axis ZL of the measurement chamber 2, and all image points 4 of the radiation detector unit 6. A patient table 12, by means of which the examination object U can be moved along the central axis ZL, is arranged in the measurement chamber 2 for the purpose of positioning the examination object U.

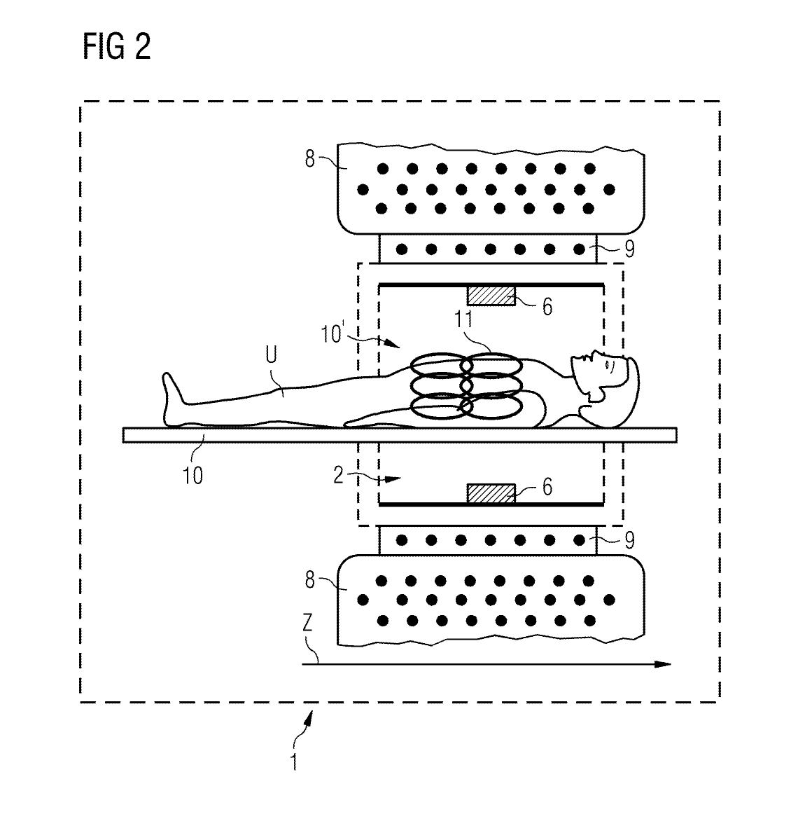

For the purpose of magnetic resonance imaging, the measurement chamber 2 of the combination imaging system 1 is surrounded by a superconducting basic field magnet 8 which generates a homogenous basic magnetic field that is oriented in the z-direction in the measurement chamber 2. The actual measurement region of the examination object U should then be situated within a homogeneity volume of the basic magnetic field as clearly shown in FIG. 2 in particular. In addition to further components known to a person skilled in the art, the combination imaging system 1 features a transmit coil, which is usually a body coil that is permanently installed around the measurement chamber in the device, and by means of which high-frequency signals can be transmitted at the desired magnetic resonance frequency in order to excite the spins in a specific region of the examination object. The combination imaging system 1 further comprises a gradient coil system 9, by means of which the spatial resolution of magnetic resonance information can be achieved. The magnetic resonance information, i.e. the magnetic resonance signals that are excited in the examination object, are usually captured by means of local coils 11 in this case. In addition to this, the local coils 11 can also be configured to generate HF fields that are used to excite the spins, and/or the resulting magnetic resonance signals can be captured by means of the body coil.

It is also clear from FIG. 2 that the combination imaging system 1 is allocated a plurality of equipment objects 10, 10′ that are required for the operation of the combination imaging system 1, these being arranged between the PET detector 6 and the examination object U during the operation of the combination imaging system 1. In particular, this relates to the equipment objects 10, 10′ for the operation the magnetic resonance imaging device 7, e.g. the local coils 11 or the patient table 12.

These equipment objects 10, 10′ change, absorb and/or scatter the photons that are produced during the electron/positron recombinations of the tracer, such that an inverse calculation of the condition of the examination object is corrupted or an evaluation of the image information is hampered by significant losses.

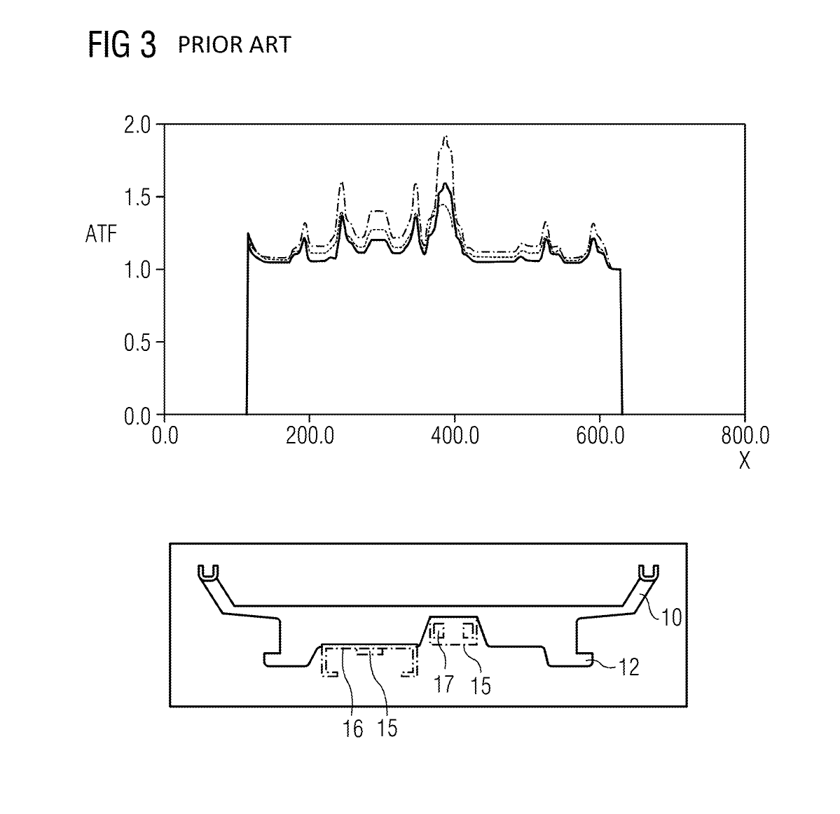

A measure of these losses is the so-called “attenuation correction factor”, the determination of which is explained in FIG. 3 with reference to a so-called “μ-map”. For this purpose, a count rate of radionuclide emission radiation (the count rate corresponds to a radiation density of the radionuclide emission radiation per image point) is first determined using a phantom radiation source U, the equipment object 10 being arranged in an operating position. The attenuation correction factor can then be determined by means of a comparison measurement in which the equipment object 10 is removed from the measurement chamber 2. The attenuation correction factor, in particular determined for each line of response, represents a scaling value by which the count rate must be multiplied in order to obtain the value of the comparison measurement. Otherwise stated, this means that the higher the determined attenuation correction factor, the lower the transmission of radionuclide emission radiation and the more adversely the radionuclide-based imaging can be affected.

In FIG. 3, a diagram above a patient table 12 that is illustrated in outline in the lower section of the image, i.e. the associated “μ-map”, therefore shows the spatial assignment of the attenuation correction factor ATF (a dimensionless scaling factor) to image points of the PET detector 6 along a line running transversely through the patient table (in an x-direction with units in mm) for “lines of response” that are perpendicular relative to this line. It can be seen from the spatial assignment that the dash-dot marked function components 15 of the patient table 12 require the highest attenuation correction factors. A gear rack made of metal with an associated bearing rail 16 for moving the patient table 12, for example, results in a peak value of the attenuation correction factor of approximately 1.5 during the operation of the respective combination imaging system 1. The centrally arranged electronics channel 17, comprising a multiplicity of metallic leads, circuit boards, sheath wave traps and other shielding devices for HF radiation, produces an even higher peak value of the attenuation correction factor of approximately 1.9. This means that nearly 50% of the radionuclide emission radiation arriving at this part is absorbed or scattered.

With regard to the cited function components 15 arranged in the central region, it can also be seen that the attenuation correction factor for a plurality of contiguous image points reaches the peak values that have been specified for the respective function component 15. For example, a significant influence on the radionuclide-based imaging can be expected if an attenuation correction factor limit value is exceeded for a predetermined number of contiguous image points (described above), wherein this occurs in the case of the function components 15 arranged in the central region, and therefore these function components are classified as “image-critical”.

In this case, equipment objects 10 such as e.g. local coils comprising function components 15 which have an attenuation value that reaches or exceeds an attenuation limit value in respect of a defined minimum cross-sectional area can result in the specified correction limit value being reached or exceeded and the shading of a PET detector region with the predetermined number of image points, or in the attenuation correction factor limit value for the predetermined number of image points being exceeded.

The attenuation values relating to the associated minimum cross-sectional areas are determined as described above in this case.

With reference to a plurality of examples of typical function components in routinely used equipment objects, said examples illustrating the principle particularly well, it is shown below how the number of contiguous image points 4 of the PET detector 6 reaching the correction limit value as specified above can be minimized by changing the design of the equipment object, such that the radionuclide-based imaging in the combination imaging system 1 is improved overall.

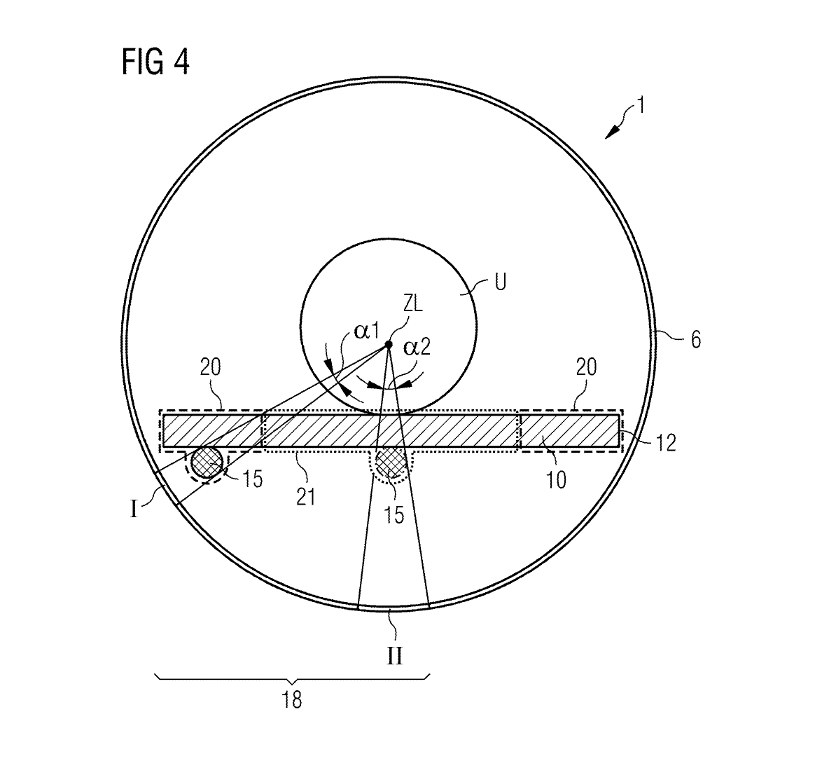

FIG. 4 schematically illustrates a first possibility whereby this can be realized. In the example embodiment, an examination object U in the form of a cylindrical phantom is arranged on the central axis ZL of the combination imaging system 1. A patient table 12 features a first image-critical function component 15 in a peripheral region 20 of the patient table 12 on the underside of the patient table 12, and a further, second image-critical function component 15 is arranged in a central region of the patient table 12, likewise on the underside of the patient table 12. The peripheral region 20 in this case immediately adjoins that narrow side or longitudinal edge of the patient table 12 coming closest to the PET detector 6, and encompasses a spatial region corresponding to the specified fraction as described above of the volume of the patient table 12.

Both function components 15 are identically configured, particularly in terms of their material composition and their dimensions. The identical function components in the combination imaging system 1 require an attenuation correction factor exceeding the correction limit value. The first function component 15, arranged in the peripheral region 20, covers an angular range relative to the PET detector 6 which is described by a first shade angle α1. Said first shade angle α1 corresponds to the shade surface I on the PET detector 6.

Similarly, a second shade angle α2 and a second shade surface II are covered by the centrally arranged second identical function component 15.

It can be seen in this case that the first shade surface I is smaller than the second shade surface II, and therefore the first shade surface I overlaps fewer image points of a PET detector 6 in a contiguous region than the second shade surface II. This is confirmed by a corresponding comparison measurement of count rates, in which only the first shade surface I or the second shade surface II respectively was covered by the identical first or second function component 15. In a ten-minute measurement illustrating the principle, the count rates were determined in each case for the PET radiation of the phantom by means of the PET detector 6. A count rate of 970630086 photons was produced when the shade surface I was covered by the function component 15 and a count rate of 97436215 photons was produced when the shade surface II was covered by the function component 15. Relative to a count rate of 97585988 photons that was determined without the first or second function component, a percental attenuation value of only 0.15% is produced for the first shade surface I while a percental attenuation value of 0.54% is produced for the second shade surface II. The inventive positioning of the identical function component 15 in the peripheral region 20 instead of arranging it in an average central region 21 of the patient table 12 therefore significantly improves the radionuclide-based image information.

This idea can be applied in a method for designing the equipment object 10 or for designing a combination imaging system 1, for example. The design comprises both the planning of the equipment object and its production in this case.

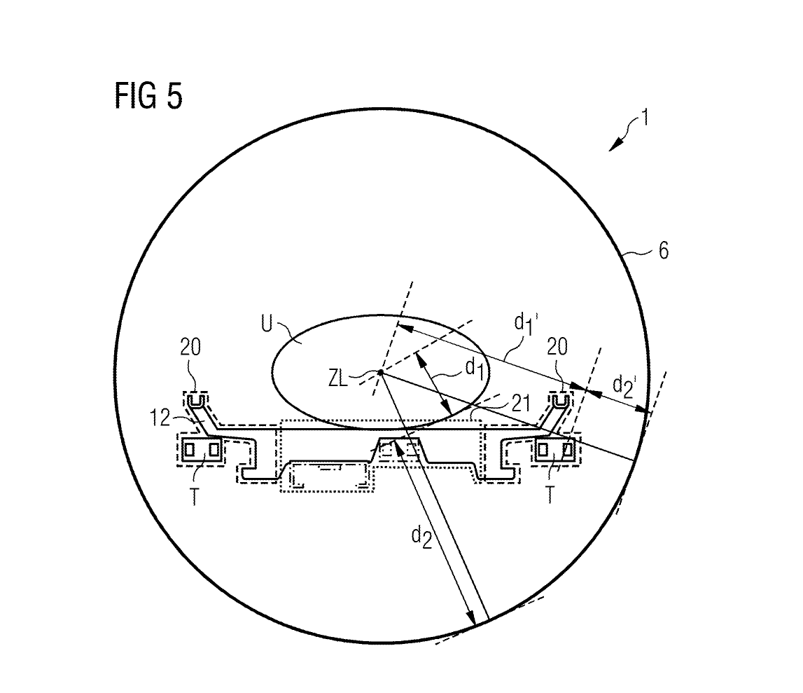

For this purpose, FIG. 5 again shows the equipment object 10 that was already illustrated in FIG. 3, namely the mobile part of the patient table 12, which is arranged in the combination imaging system as shown schematically in FIG. 2.

In a first step of an embodiment of the inventive design method, provision is made for identifying and selecting image-critical function components 15 illustrated in this example embodiment. This selection is made on the basis of the attenuation value of the function components 15 relative to an effective minimum cross-sectional area assigned to the attenuation value, or on the basis of the above cited other parameter combinations which were described previously for the purpose of identifying an image-critical function component 15. In particular, it may relate to an instance of the attenuation correction factor exceeding the stipulated correction limit value, preferably in respect of the predetermined number of contiguous image points of the PET detector 6, or it may relate to the shade surface and/or the shade angle which is produced by the image-critical function component 15 in respect of radionuclide emission radiation relative to the PET detector. In this case, the selection and identification of the image-critical function components 15 can already take place in the planning phase, e.g. on the basis of existing knowledge from previous sample measurements or theoretical calculations and/or by means of simulations. In order to achieve this, it is not necessary first of all to actually produce the equipment object 10 featuring the unfavorably arranged function component 15. This means that those function components 15 which are broken-marked in the figures are no longer present in the equipment objects that are produced according to an embodiment of the invention, but will only be found at these positions in the corresponding conventional equipment objects according to the prior art. A conventional patient table 12 normally features a plurality of image-critical function components 15, i.e. the electronics channel 17 and the bearing rail 16 in this case. At least one of these image-critical function components 15 of different types is selected for the purpose of optimizing its influence on the radionuclide-based imaging.

In the example embodiment according to FIG. 5, the electronics channel 17 is selected first for optimization. The electronics channel 17 contains a sheath wave trap, a number of cables and a plurality of further electronic or electrical components which have a significant metallic portion and therefore a high attenuation value for radionuclide emission radiation. In the case of the electronics channel 17, the surface portion occupied by metallic components is between 5% and 15% on a plane which is parallel with the table surface of the patient table 12 facing the examination object (relative to the overall surface occupied by the electronics channel on this plane).

In the example embodiment, the electronics channel 17 is arranged in a peripheral region 20 of the equipment object 10 in accordance with a further step of the design method. For example, this can be at one of the positions designated T in the lateral peripheral region 20 of the patient table 12 in FIG. 5, immediately adjacent to the narrow side of the essentially flat patient table 12. Arranged in the position T, the electronics channel 17 forms a spatial extension along the lateral narrow side of the patient table 12 and at the same time forms a periphery, directly facing the PET detector 6, of the patient table 12.

As a result of the transfer from the conventional position in the central region 21 to the peripheral position T, the distance d1′ between the electronics channel 17 and the central axis ZL of the measurement chamber 2 is now greater than the distance d1 in the previous design, while the distance d2 between the electronics channel 17 and the PET detector 6 is reduced to a smaller distance d2′ at the same time. In the peripheral region 20, the electronics channel 17 is therefore located at a position which essentially corresponds to a minimal distance from the closest surface of the PET detector 6, while the distance of the electronics channel 17 from the central axis ZL of the measurement chamber 2 is essentially maximized at the same time. The term “essentially” in this context is understood to mean that the minimal distance between the equipment object and the surface of the PET detector 6 differs from the distance between the electronics channel 17 and the surface of the PET detector 6 by only the thickness of the boundary wall.

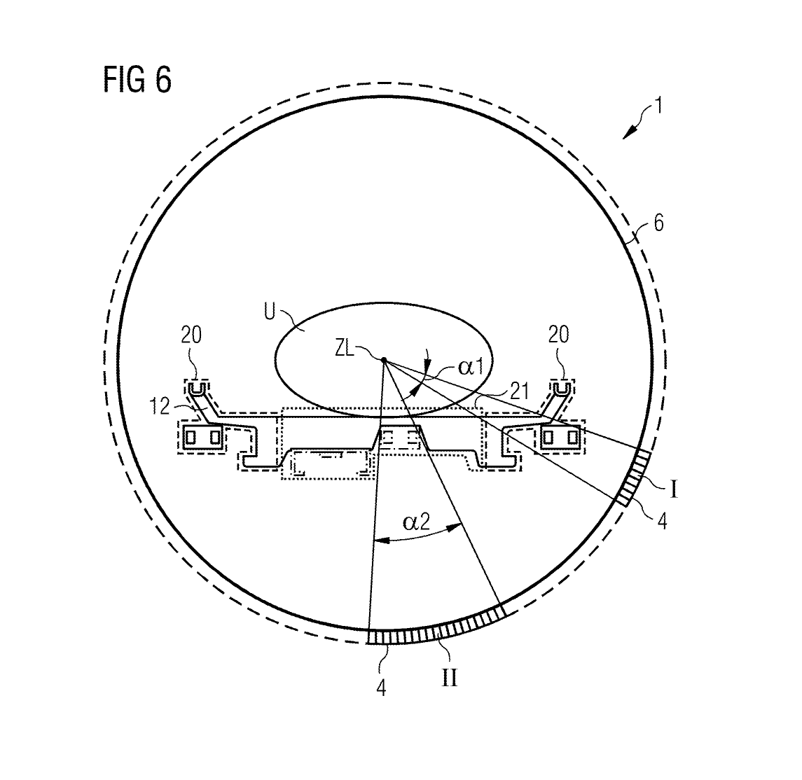

FIG. 6 shows the associated shade angles α1, α2 of the electronics channel 17 for a position in the peripheral region 20 and a position in the spatially central region 21 of the patient table 12. The shade angle α1 for the position T in the peripheral region 20 is clearly smaller than the shade angle α2 for the position of the electronics channel 17 in the spatially central region 21. The shade surface I associated with the smaller shade angle α1 therefore overlaps fewer contiguous image points 4 on the surface of the annular PET detector 6 than the shade surface II associated with the larger shade angle α2, such that the field of view for the PET imaging is improved thereby.

According to an embodiment of the design method, the electronics channel 17 can be “repositioned” or transferred into the peripheral region 20, preferably parallel with the face side of the patient table 12, until the shade surface I reaches or falls below a number, this being predetermined as described above, of contiguous image points 4 of the PET detector 6.

As indicated above, it can be taken into consideration by an example embodiment of the invention that only those “lines of response” running through the examination object and representing so-called relevant “lines of response” contribute to the radionuclide-based imaging.

In this case, the peripheral region 20 of the patient table 12 in which it is acceptable to arrange image-critical function components according to an embodiment of the invention can alternatively be determined by an optimal position relative to the relevant “lines of response”, and then comprises all of the positions lying outside of the projection of the examination object onto the detector surface and parallel with the support surface on the patient table. In this case, the projection of a contour of a typical patient onto the support surface of the patient table then defines the spatially central region 21 of the equipment object accordingly.

This might mean that the image-critical function components can be arranged significantly closer to the center of the patient table 12 in that region of the patient table which is intended for supporting the head than in the region of the torso, since the spatially central region 21 has significantly smaller dimensions in the region of the head.

As indicated above, the group of image-critical function components 15 of the patient table 12 also includes the bearing rail 16 for moving the patient table 12 in the measurement chamber 2. In order to optimize the arrangement of the bearing rail 16 relative to the field of view of the PET detector 6, the design can now allow for the gear rack, which is made entirely of metal, and the associated bearing rail 16, which is likewise made of metal, to be transferred parallel with the face side (i.e. the upper side or underside or patient support surface) of the patient table 12 into the peripheral region 20 thereof in accordance with the method described above. However, the shade surface is still significant due to the spatial breadth of the bearing rail 16 in the plane of the outward transfer, and therefore there may remain scope for improvement in respect of the “field of view” of the detector even after arrangement in the peripheral region 20.

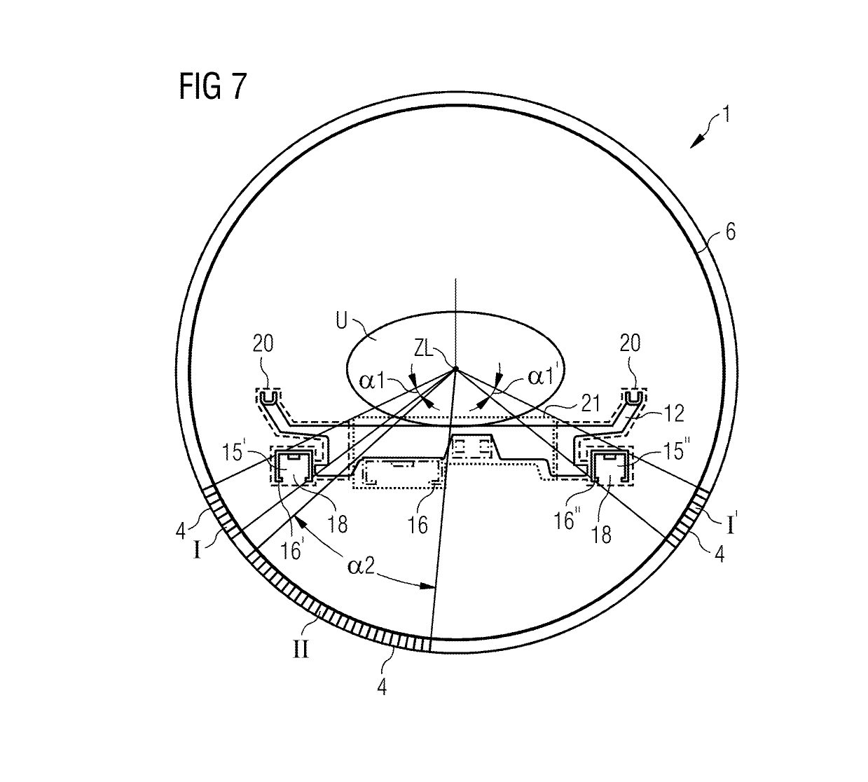

In an alternative form as illustrated in FIG. 7 of the method for designing the patient table 12, provision is therefore made for partitioning the bearing rail 16 into a function assembly 18 consisting of first and second function components 15′, 15″ in the form of partial bearing rails 16a and 16b, which together have the functionality and overall capacity of the original bearing rail 16. The partial bearing rails 16a, 16b are essentially identical in function, but are significantly more compact, particularly in the direction of the transfer, i.e. in a breadth direction parallel with the table surface or support surface, than the original bearing rail 16.

As an alternative to the example embodiment illustrated here, the partitioning of the bearing rail 16 is not restricted to a first and a second partial bearing rail 16a, 16b in this case. In addition to allowing for functional considerations such as e.g. the overall capacity and overall functionality, the partitioning of the function component 15 can also provide for the partial bearing rails 16a, 16b to be optimally dimensioned in relation to the field of view. This means that the bearing rail 16 can be repeatedly partitioned until each partial bearing rail 16a, 16b only covers at most the predetermined number of contiguous image points of the PET detector 6.

It is also evident from FIG. 7 that the partial bearing rails 16a, 16b are spatially separate from each other and, forming a section of one of the opposing narrow sides of the table 12, are arranged as a continuation of the lower face side of the patient table 12, such that said lower face side is extended in the direction of the narrow side of the patient table 12 by these partial bearing rails 16a, 16b. The dimension of the patient table 12 is therefore increased in the direction of the closest surface of the PET detector 6, such that the external extent of the patient table 12 increases as a result of the arrangement of the bearing rail 16 being optimized in respect of the field of view of the PET detector 6.

It is also readily apparent from FIG. 7 that the shade surfaces I and I′ associated respectively with the partial bearing rails 16a and 16b, together advantageously represent a smaller total shade surface than the shade surface II which is produced by the arrangement of the bearing rail 16 in the original central position shown, such that the desired improvement in respect of the field of view of the PET detector 6 is achieved here too.

As a result of transferring the electronics channel 17 and the bearing rail 16 into the peripheral region 20 of the patient table 12, the central region 21 of the inventive patient table is free of function components 15 which exceed the attenuation limit value, and therefore a specified central limit value of the attenuation value is no longer exceeded anywhere in the central region 21 in the example embodiment. Likewise, the central region 21 is free of function components 15 which exceed the central correction limit value for the predetermined number of image points.

As an alternative or in combination with the above, in addition to the cited function components (bearing rail 16 and electronics channel 17), strengthening structures in the patient table 12 can also be transferred in such a way that the desired central limit value in the central region 21 is satisfied or not reached. Said strengthening structures may comprise e.g. ridges in a flat equipment object, which run from one face side to an opposite face side.

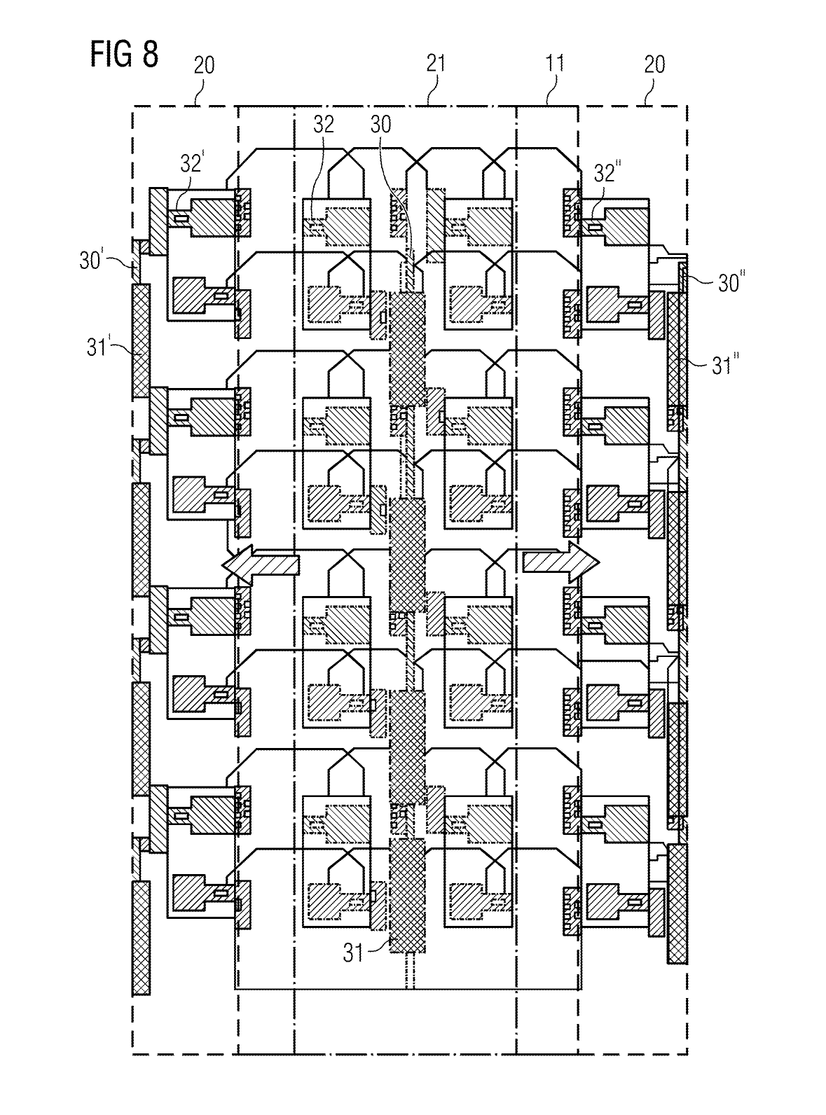

FIG. 8 shows a further example embodiment of the invention, in which this idea is applied. In order to depict the spinal column during operation of the combination imaging system 1, the essentially flat local coil 11 (spine coil) is arranged as standard to lie with its lower face side (local coil underside) in a recess on the upper face side of the patient table 12. The illustration in FIG. 8 shows the local coil 11 in a plan view of its almost rectangular face side. In the case of a standard arrangement of the local coil 11 in the measurement chamber of the combination imaging system, the lengthwise direction of the rectangular face side corresponds to the direction z of the basic magnetic field of the combination imaging system.

In a spatially central region 21 of an original (i.e. not inventive) starting design, an elongated multilayer printed circuit board 30 having a length of approximately 110 cm and being oriented in the z-direction is arranged in the center or in a central region of the local coil 11 (delimited by a broken line in FIG. 8). All of the connection lines to individual elements of the local coil 11 (e.g. to antenna elements or preamplifier units) are grouped in or on this multilayer printed circuit board 30. Said printed circuit board 30 is shielded on its outer layer. Provision is also made for mounting so-called bazookas 31 on the printed circuit board 30. These are CU-coated cuboids made of MR-suppressing plastic, which are usually clipped onto the printed circuit board 30 at intervals of 20 cm, i.e. approximately one fifth of the length of the printed circuit board 30.

The bazookas 31 and the printed circuit board 30 require the highest attenuation correction factors of all the function components of the local coil 11 in the combination imaging system, representing significant image-critical function components 15, and therefore should be arranged in a peripheral region 20 of the local coil 11 in accordance with an embodiment of the invention, in order that they are located in an optimized position in respect of the field of view of the PET detector (not shown in FIG. 8).

These function components 15 are therefore already transferred into the broken-marked lateral peripheral region 20 of the local coil 11 which directly adjoins the narrow side of the local coil 11, as indicated schematically by means of arrows, during the design and/or production of the local coil 11 in the example embodiment.

The arrangement of the image-critical function components in the peripheral region 20 is preferably effected from the outside inwards, in the order of the maximal attenuation correction factor produced by the relevant function components, i.e. the more image-critical the function components in particular individually are considered to be (determined either on the basis of the surface of the function component, the attenuation correction factor, the shade angle, the attenuation value or any desired combination of these measures), the further outwards they are transferred in the peripheral region 20.

Therefore the bazookas 31 and the elongated printed circuit board 30, requiring as they do the highest attenuation correction factor, are arranged as far away as possible in the outermost peripheral region 20 of the local coil 11, i.e. directly adjacent to the peripheral edge of the local coil 11.

The local coil 11 additionally comprises further circuit board elements 32 bearing electrical modules, in particular discrete modules, preferably for tuning devices, or also integrated modules such as amplifier circuits, for example. These circuit boards or electrical modules in the example embodiment have a lower maximal attenuation correction factor than the elongated circuit board 30 and the bazookas 31 and, though still arranged in the peripheral region 20, are therefore arranged further inwards or further away from the peripheral edge of the local coil 11 than the elongated circuit board 30 and the bazookas, in accordance with the order of the maximal attenuation correction factor. The order in which the function components are arranged in the peripheral region 20 of the local coil is therefore selected such that the maximal attenuation correction factors of the respective function components decrease as the distance from the edge of the local coil 11 and the proximity to the central region 21 of the local coil increase.

In this case, the arrangement of the function components in a peripheral region 20 in the order of the attenuation correction factors again optimizes the field of view of the PET detector relative to these function components as a complete arrangement, thereby providing an improved PET-based representation of the examination object overall.

Alternatively, the order of the arrangement of the critical function components can also be defined by the cross-sectional area of the function components, by the number of image points that are overlapped by the shade surface of the respective function component, by the attenuation value of the function component, or by a combination of these parameters.

It is also evident from FIG. 8 that the elongated printed circuit board 30 and the bazookas 31 are partitioned to form a function assembly 18 in this example embodiment. Both the elongated printed circuit board 30 and the bazookas 31 are arranged as essentially functionally identical first and second partial function components on opposite longitudinal sides of the local coil 11 and are reduced in width relative to the original function component.

The first function component in this case represents the combination of a first elongated printed circuit board 30′ and first bazookas 31′, said combination being arranged in the left-hand peripheral region 20 of the local coil 11 in the illustration. The second function component corresponds to a functionally identical and essentially equally dimensioned combination of second printed circuit board 30″ and second bazookas 31″, said second combination being arranged in the right-hand peripheral region 20 of the local coil 11 in FIG. 8. In this case, “essentially equally dimensioned” is understood to mean that the first and second function components can be inscribed into an identical cuboid, wherein each side surface of the cuboid corresponds at least at one point to a peripheral surface of the respective first or second function component. The same applies to the associated circuit boards 32, which are designated 32′ in the optimized position on the left-hand periphery and 32″ on the right-hand periphery.

The connection lines to the individual elements of the local coil 11 are no longer grouped in a single elongated printed circuit board 30 in this example embodiment. Instead, the printed circuit boards 30′ and 30″ arranged in the left-hand and right-hand peripheral region 20 of the local coil 11 form a function assembly 18, which in combination forms the connection to the individual elements. The spatially central region 21 of the local coil 11, as identified by the dash-dot outline, is therefore free of corresponding image-critical function components and has an attenuation value which does not reach the central attenuation limit value anywhere in said region.

By virtue of reducing the dimensions of the respective printed circuit boards 30′ and 30″ relative to a centrally arranged printed circuit board 30 and transferring the printed circuit boards 30′ and 30″ which are configured as a function assembly 18 into opposing peripheral regions 20 in the direction of the narrow sides of the local coil 11, as explained above with reference to the bearing rail for a patient table, the field of view of the PET detector is again optimized. In this example embodiment likewise, the transfer of the critical components requires an extension of the external extent of the local coil 11 and in particular the face side of the local coil. In the plan view of the face side of the local coil 11, the critical function components are not overlapped by any further function components of the local coil 11 with the exception of a housing or casing, nor do they overlap any further function components, and in this sense the critical function components can therefore be described as being arranged separately in the equipment object, in a peripheral region 20 of the local coil 11.

It is clear from the foregoing description that at least one embodiment of the invention offers effective possibilities for reducing any interference or changes in respect of radionuclide-based image information in a combination imaging system.

It should be noted in this case that the features of all example embodiments or developments disclosed in the figures can be used in any combination. Attention is likewise drawn to the fact that the equipment objects, combination imaging systems and methods for designing an equipment object as described in detail above are merely example embodiments, which can be modified in all variety of ways by a person skilled in the art without thereby departing from the scope of the invention. Furthermore, use of the indefinite article “a” or “an” does not exclude the possibility of multiple occurrences of the features concerned.

What is claimed is:

1. An equipment object, positionable in a measurement chamber and provided for a combination imaging system including a radionuclide imaging device and a magnetic resonance imaging device, the equipment object at least one of:
comprising an image-critical function component, in a peripheral region of the equipment object, including an average radionuclide emission radiation attenuation value that reaches at least a specified attenuation limit value of 30% in relation to a first defined minimum cross-sectional area of 30 mm²; and
configured such that an average radionuclide emission radiation attenuation value relating to a second defined minimum cross-sectional area of 400 mm² of the equipment object reaches at most a central attenuation limit value of 15% in an overall spatially central region of the equipment object.

2. The equipment object of claim 1, wherein the image-critical function component includes metallic portions whose cross-sectional area represents at least 20% of the first minimum cross-sectional area.

3. The equipment object of claim 1, wherein the equipment object comprises a plurality of the image-critical function components in the peripheral region of the equipment object and, on opposite sides thereof, said image-critical function components at least one of are essentially identical in function and have an overall functionality that results from the combination of the function components.

4. The equipment object of claim 1, wherein the equipment object is so configured as to be essentially flat.

5. The equipment object of claim 1, wherein the equipment object comprises at least one of a device item and a function ancillary unit, for at least one of receiving and exciting magnetic resonance signals.

6. The equipment object of claim 1, wherein the image-critical function component is selected from a group of at least one of
mechanical components comprising at least one of mechanical drive components, guide components, and strengthening components, and/or
electrical components comprising at least one of shielding devices, bazookas, circuit boards, cable sections, electrical modules, and integrated modules.

7. A combination imaging system, comprising:
a radionuclide imaging device, including a radiation detector unit for radionuclide emission radiation;
a magnetic resonance imaging device; and
an equipment object, arranged in a measurement chamber of the combination imaging system between an examination object and the radiation detector unit, wherein at least one of
the equipment object comprises, in a peripheral region, an image-critical function component which requires an attenuation correction factor that reaches a correction limit value of at least 1.5, and
the equipment object, in an overall spatially central region, is so configured that an attenuation correction factor required by the central region reaches at most a central correction limit value of 1.2.

8. The combination imaging system of claim 7, wherein the image-critical function component arranged in the peripheral region of the equipment object is associated with a shade surface of the radiation detector unit, and wherein said shade surface corresponds to at least a number of image points arranged contiguously on the radiation detector unit.

9. The combination imaging system of claim 7, wherein the surface ratio of a shade surface of the image-critical function component projected onto the radiation detector unit to the projection surface of a face side of the equipment object on the radiation detector unit does not exceed 1:10.

10. A method for designing an equipment object, comprising:
identifying a first image-critical function component on the basis of at least one of its attenuation value for radionuclide emission radiation and an attenuation correction factor; and
arranging the identified first image-critical function component, or at least parts of the image-critical function component, in a peripheral region of the equipment object.

11. The method of claim 10, further comprising:
partitioning the functionality of the image-critical function component by
providing an essentially functionally identical further function component or providing two essentially functionally identical further function components, which interact in such a way that they fulfil the function that must be fulfilled by the image-critical function component during operation, and
so arranging such further function components that they are spatially separate from each other or from the first image-critical function component.

12. The method of claim 10, further comprising:
increasing the dimensions of the equipment object in a spatial direction,
arranging the image-critical function component in the region of the extension of the equipment object.

13. A method for designing a combination imaging system which includes a radionuclide imaging device and a magnetic resonance imaging device and an equipment object that is arranged as standard between an examination object and a radiation detector unit for radionuclide emission radiation, said method comprising:
identifying an image-critical function component of the equipment object on the basis of at least one of an attenuation value and an attenuation correction factor; and
arranging the identified function component or at least parts of the image-critical function component in the peripheral region of the equipment object.

14. The equipment object of claim 2, wherein the image-critical function component includes metallic portions whose cross-sectional area represents at least 30% of the first minimum cross-sectional area.

15. The equipment object of claim 14, wherein the image-critical function component includes metallic portions whose cross-sectional area represents at least 40% of the first minimum cross-sectional area.

16. The equipment object of claim 2, wherein the equipment object comprises a plurality of the image-critical function components in the peripheral region of the equipment object and, on opposite sides thereof, said image-critical function components at least one of are essentially identical in function and have an overall functionality that results from the combination of the function components.

17. The equipment object of claim 5, wherein the device item is a patient table and the function ancillary unit is a local coil.

18. A combination imaging system, comprising:
a radionuclide imaging device, including a radiation detector unit for radionuclide emission radiation;
a magnetic resonance imaging device; and
an equipment object, arranged in a measurement chamber of the combination imaging system between an examination object and the radiation detector unit, wherein at least one of
the equipment object comprises, in a peripheral region, an image-critical function component which requires an attenuation correction factor that reaches a correction limit value of at least 1.5, and
the equipment object, in an overall spatially central region, is so configured that an attenuation correction factor required by the central region reaches at most a central correction limit value of 1.2, wherein the equipment object is configured as claimed in claim 1.

19. The method of claim 11, wherein such further function components are arranged such that they are spatially separate from each other or from the first image-critical function component, on essentially opposite sides of the equipment object.

20. A method for designing the equipment object of claim 1, comprising:
identifying a first image-critical function component on the basis of at least one of its attenuation value for radionuclide emission radiation and an attenuation correction factor; and
arranging the identified first image-critical function component, or at least parts of the image-critical function component, in a peripheral region of the equipment object.
